(19) United States
(12) Patent Application Publication
(10) Pub. No.: US 2019/0234035 A1
(43) Pub. Date: Aug. 1, 2019

(54) METHOD FOR COMPACTION DETECTION AND CONTROL WHEN COMPACTING A SOIL WITH A DEEP VIBRATOR

(71) Applicant: Keller Holding GmbH, Offenbach/Main (DE)

(72) Inventors: Peter Nagy, Wien (AT); Dietmar Adam, Mödling (AT); Christoph Adam, Innsbruck (AT); Maximilian Schmitter, Kirchberg in Tirol (AT); Fritz Kopf, Wien (AT); Gerhard Strauch, Wien (AT); Peter Freitag, Eggendorf/Siedlung Maria Theresia (AT)

(21) Appl. No.: 16/256,457

(22) Filed: Jan. 24, 2019

(30) Foreign Application Priority Data
Jan. 26, 2018 (EP) EP18153596

(51) Int. Cl. E02D 3/054; E02D 3/074; B06B 1/16; G01N 3/08; G01N 33/24

(52) U.S. Cl. CPC E02D 3/054; E02D 3/074; G01N 33/24; G01N 3/08; B06B 1/164

(57) ABSTRACT

A method for detecting and controlling compaction when compacting a soil by a depth vibrator which has a rotationally drivable imbalance (3) and at least one sensor (6, 12, 13, 14, 19), comprising the steps of:
inserting the depth vibrator (2) into the soil (17) up to a desired final depth (Tm);
compaction of the the soil (17) during which the forward angle (φ) of the imbalance (3) as well as the oscillation amplitude (A) of the depth vibrator (2) are determined;
detection of a soil stiffness profile from soil stiffness values (k) determined over time (t);
determination of a first soil stiffness value (k1) and a second soil stiffness value (k2) from the soil stiffness profile (k), for which it applies that a rate of increase (k′2) of the second soil stiffness value (k2) exceeds a rate of increase (k′1) of the first soil stiffness value (k1) by a defined factor;
calculation of a transition soil stiffness value (k12) which is between the first soil stiffness value (k1) and the second soil stiffness value (k2); and
storing the transition soil stiffness value (k12) detected in the respective compaction step to the associated depth (T).

### CROSS-REFERENCE TO RELATED APPLICATION(S)

The present application derives priority from European Patent Application EP18153596 filed 26 Jan. 2018.

### BACKGROUND OF THE INVENTION

### Field of the Invention

The present invention relates to soil improvement and, more particularly, to a method for compaction detection and control when compacting a soil by a depth vibrator and a method for soil improvement using a depth vibrator that is carried out using data from said method for compaction detection and control.

### Description of the Background

For the depth compaction of loosely packed soils, depth vibrators are used which are dynamically excited by a rotating imbalance and form a dynamic interaction system with the surrounding soil to be compacted. By means of measurements on the vibrator, the dynamic excitation, especially the imbalance and its position, as well as the vibrator motion can be determined, and from this the dynamic properties of the surrounding soil can be determined. Depth vibrators can be used in various soil improvement techniques, as described, for example, in the applicant's brochure “Die Tiefenruttelverfahren” (Prospekt 10-02D).

The compaction of the soil is usually performed by the dynamically excited vibrator penetrating into the soil at a compaction point to the desired compaction depth, if necessary with the aid of water flushing, and then either being drawn in steps and respectively held to a termination criterion, or by tamping movements in an upwardly moving pilger process. Compaction over large areas is achieved by processing many compaction points in one grid.

From WO 2012/171527 a method for soil sounding and a method for producing material columns in the soil after soil sounding is known. The method comprising the steps of: insertion of a vibrator arrangement to a predetermined depth into the ground; determination of a ground profile of the ground upon insertion of the vibrator arrangement, wherein determination of the ground profile comprises measuring at least one operating parameter of the vibrator arrangement upon insertion into the ground, and wherein the ground profile each comprises a ground parameter for at least two different ground depths. The operating parameters that can be measured are the power consumption of the vibrator motor while insertion of the vibrator arrangement into the ground, the speed at which the vibrator arrangement is inserted into the ground, or a vibration amplitude of the tip of the vibrator arrangement.

From DE 199 28 692 a method for online compaction control of a soil when using a depth vibrator is known. The method provides that for the measured penetration depth of the depth vibrator, the tilt angle to the vertical zero axis, the leading angle of the imbalance relative to the vibrator shell and at least one horizontal deflection between the vibrator tip and the vibrator joint is directly or indirectly detected by one or more sensors mounted on or in the vibrator tip. From these recorded sensor data, soil dynamic characteristic values of the vibrator-soil system such as damping and spring stiffness are calculated and from this the momentary packing density of the surrounding soil is determined. This value determined for the packing density packing density is constantly compared with a given target value for the packing density of the soil and the vibration process is continued until the target value for the packing density of the soil is reached.

From DE 101 46 342 a method for determination of the packing density of soil during soil improvement by vibration pressure compaction is known. A depth vibrator with an imbalance rotating about a vertical axis hanging from a rod, is vibrated into the ground, if necessary with the addition of water and/or air, pulled in individual height layers and held vibrating in individual depth positions at intervals of vibration. The packing density is calculated from the amplitude of the vibrator under consideration of stress dependent soil parameters by a computer or processor unit.

From EP 1516961 B1 a method for determination of the soil stiffness of a compacted or to be compacted soil area and a soil compaction device is known. The soil stiffness is determined from vibrations introduced into the soil, from the machine parameters of the soil compaction device and from the temporal position of the soil compaction force.

The present invention is a method for online compaction detection and control that enables efficient soil compaction by a depth vibrator. The task also consists of proposing an appropriate method for improving a ground by a depth vibrator, which is carried out on the basis of said method for online compaction detection and control.

### SUMMARY OF THE INVENTION

Accordingly, it is an object of the invention to provide an improved method for compaction detection and control when compacting a soil using a depth vibrator. It is another object to provide an improved method for soil improvement using a depth vibrator that is carried out using data from said method for compaction detection and control. The vibrator is of the type that uses a rotational imbalance, e.g., a rotational offset weight. The method includes the steps of:
inserting the depth vibrator into the soil up to a desired final depth (Tm);
compaction of the soil during which the forward angle (φ) of the imbalance as well as the oscillation amplitude (A) of the depth vibrator are determined;
detection of a soil stiffness profile from soil stiffness values (k) determined over time (t);
[Note: compaction of the soil around the vibrator increases its stiffness. The soil stiffness is determined by accompanying measurements during the compaction process. This means that the respective physical quantities and/or characteristic values are recorded or calculated over time. This can take place continuously respectively at regular or irregular intervals. Preferably, in order to determine the soil stiffness values above the depth of the compaction bodies to be created, the vibrator is gradually pulled upwards in individual compaction steps starting from the final depth. The individual compaction steps each refer to an associated compaction depth, also referred to as the compaction point, to which the vibrator is brought to compact this area of soil. A soil stiffness profile can be created on the basis of all or at least a partial number of the transition soil stiffness values recorded above depth (T).]
determination of a first soil stiffness value (k1) and a second soil stiffness value (k2) from the soil stiffness profile (k) at which a rate of increase (k′2) at the second soil stiffness value (k2) exceeds a rate of increase (k′1) at the first soil stiffness value (k1) by a defined factor.
[Note; given the soil stiffness profile in which soil stiffness is determined over time for at least one part of, in particular the largest part of, preferably all compaction depths, the development of the soil stiffness (rate of increase) is established and can be used to document the compaction success, to optimize the process parameters, the compaction sequence, the selected compaction grid or to automatically control the vibrator.]

For example, in an embodiment the foregoing data is used to calculate a transition value representing the point at which soil stiffness suddenly increases. In this case the method additionally includes the steps of:
calculation of a transition soil stiffness value (k12) which is between the first soil stiffness value (k1) and the second soil stiffness value (k2); and
storing the transition soil stiffness value (k12) detected in the respective compaction step to the associated depth (T).
[Note: the increase in soil stiffness—as long as the soil is further compacted—is at least essentially constant. The formulation “essentially constant” is intended to take particular account of the fact that measurement inaccuracies and soil irregularities can lead to deviations. If the soil is as compacted as possible, further vibrating will lead to a noticeable increase in soil stiffness. The function of the soil stiffness determined over time makes a noticeable kink. This can be explained in particular by the fact that when this maximum compaction state is reached, no further compaction takes place with further vibration, but only grain-on-grain compaction and/or grain breakage. The sudden increase in soil stiffness or the buckling in the graph can be determined in any suitable way. For example, a first soil stiffness value at a first point in time and a second soil stiffness value at a later second point in time may be determined such that the rate of increase associated with the second soil stiffness value is significantly greater than the first rate of increase in soil stiffness. A time interval between the first time and the second time may be chosen in an appropriate manner and may be, for example, less than 10% and/or more than 1% of the duration of the compaction operation at a compaction depth. From the determined first and second soil stiffness value a transition soil stiffness value can be calculated, which is between the mentioned values. The transition value represents the point at which soil stiffness suddenly increases. Alternatively or in addition, the transition soil stiffness value can be determined by regression analysis of the soil stiffness curve, in particular by mathematical decomposition into two lines of regression. The intersection of the regression lines can be calculated as a transition value-soil stiffness value. Another possibility is to derive soil stiffness increase rates (k′) from the soil stiffnesses (k) continuously determined at a compaction depth, wherein a first soil stiffness increase rate (k′1) can be compared with at least one second soil stiffness increase rate (k′2) determined at a later time and a transition soil stiffness value (k12) for the respective compaction depth can be determined therefrom if the second soil stiffness increase rate (k′2) exceeds the first soil stiffness increase rate (k′1) by a defined factor.

One advantage of the online compaction control method is that it reliably detects when the soil is no longer compacted or hardly compacted at all. This is done by determination of the soil stiffness over time within a compaction depth during the compaction process. The slope of the soil stiffness can be derived from two or more determined soil stiffness values. It is intended that the soil stiffness profile will be used for the construction of other compaction bodies. The compaction process respectively the vibrating in at a certain depth can be terminated if the determined soil stiffness suddenly increases, i.e. the determined soil stiffness increases more strongly over time than with previously determined soil stiffnesses, or the transition soil stiffness value associated with the respective depth is reached.

By using the soil stiffness monitored over time as a criterion for terminating compaction at a given depth, a maximum degree of compaction is achieved with maximum efficiency. By stopping the vibration when a significant increase in soil stiffness is achieved, maximum compaction can be achieved in minimum vibration time. It is avoided that unnecessary time is spent on vibrating without significant compaction success. Furthermore, the wear on the depth vibrator, which increases with increasing degree of compaction, e.g. due to grain distortion at the vibrator tip, can be significantly reduced.

After a possible concretization, the compaction can in particular be stopped at the respective compaction depth if the determined second soil stiffness increase rate is greater than 1.5 times one or more soil stiffness increase rates determined at a previous time, in particular greater than 2.0 times of one or more soil stiffness increase rates determined at a previous time. A significant increase in soil stiffness determined in this way allows reliable conclusions to be drawn about the maximum compaction achieved, or at least the compaction achieved to the greatest possible extent.

According to a method implementation it is provided that the depth vibrator is first vibrated into the soil up to the desired final depth, if necessary by adding water. The depth vibrator is then pulled into steps to compact the soil step by step from bottom to top. According to a first possibility the depth vibrator can be held at the respective compaction depth after pulling to the next stage. Alternatively, the depth vibrator can also be vibrated into deeper ground areas under tamping movements, which is also referred to as the pilger process. During gradual compaction the stiffness parameter k, representing the soil stiffness, is determined relative to the depth of the compaction column, resulting in an overall stiffness profile of the soil relative to the depth. The stiffness profile can, for example, be stored using a suitable data memory and used for the compaction of further columns in a compaction field.

According to a possible embodiment, several sensors can be attached to the depth vibrator, which sense the motion behaviour or the acceleration and/or the position of the unbalance. Preferably several acceleration sensors in different planes are attached to the depth vibrator. The stepwise pulling of the depth vibrator particularly can be selected in relation to the position of the planes of the acceleration sensors in such a way that at least one of the acceleration sensors, or the associated plane, of an upper compaction depth (T) lies within the depth section defined between the planes during the previous compaction of the underlying compaction depth.

Bidirectional acceleration measurements in two planes of the vibrator can be used to determine the horizontal motion behaviour at any point of the vibrator, for example at the tip or the position of the unbalance excitation. By integrating these horizontal accelerations twice, the corresponding oscillation paths can be determined, which lead to the oscillation path amplitude A when halved. The vibrator motion is lagging behind the causing dynamic excitation by the rotationally driven unbalance. By determination of the position of the unbalance and comparing it with the vibrator motion, the feed angle φ can be determined.

The soil stiffness is calculated in particular taking into account the forward angle (φ). The forward angle (φ) describes the phase angle by which the unbalance mass is offset from the measuring direction of the sensors during the vibration motion. In addition, the soil stiffness signal (k) representing the soil stiffness of the soil can also be calculated taking into account the mass (M) of the depth vibrator. Alternatively or in addition, the determination of the soil stiffness signal (k) representing the soil stiffness of the soil can be carried out taking into account a soil mass characteristic value (ΔM) representing the soil mass resonating at the depth vibrator, in particular the modal soil mass resonating at the same time. This soil mass characteristic value (ΔM) representing the resonating soil mass can be calculated, for example, on the basis of the unbalance (m·e), the vibration amplitude (A) and the mass (M) of the depth vibrator, wherein the calculation in particular is at least approximately according to the formula:

$$Δ M = \frac{m \cdot e}{A} - M$$

According to a preferred method implementation, the determination of the soil stiffness signal (k) representing the soil stiffness of the soil can also be performed under consideration of the measured amplitude (A) and a reference amplitude (A∞). The amplitude of the vibrator at a certain excitation frequency (w) when free oscillating can be used as a reference amplitude. In order to calculate the amplification function (V), the measured vibration displacement amplitude (A) can be adjusted to the theoretical amplitude (A∞) at theoretically infinitely high excitation frequency, i.e.

$$V = \frac{A}{{A}_{∞}} = \frac{A \cdot ( M + Δ M )}{m \cdot e}$$

where M is the modal vibrator mass, ΔM the modal resonating soil mass and m·e the unbalance of the vibrator (unbalance mass times eccentricity). For free oscillation, the amplification factor is one.

According to a possible method, the determination of the soil stiffness signal (k) representing the soil stiffness of the soil can at least approximately be carried out according to the formula:

$$k = m \cdot e \cdot {ω}^{2} \cdot ( \frac{1}{{A}_{∞}} - \frac{1}{A} \cdot \frac{sign ( ϕ - 90 ° )}{\sqrt{1 + {tan}^{2} ϕ}} )$$

where
A is the vibration amplitude of the depth vibrator during compaction,
A∞ the vibration amplitude of the depth vibrator when free oscillating or with an excitation frequency towards infinity,
F is the centrifugal force,
m is the unbalance mass,
e is the eccentricity of the rotating unbalance m relative to the axis of rotation,
ω is the angular velocity of the rotating unbalance, and
   φ the phase advance of the rotating unbalance m to the mass M of the depth vibrator. This formula also takes into account the dynamic portion of the vibration-relevant quantities. It is understood, however, that in principle other calculation methods are also possible for the calculation of a stiffness signal representing the stiffness of the soil.

The task is also solved by a method to improve a ground using a depth vibrator, with the steps: Creating a first compaction body, wherein the depth vibrator is inserted into the ground up to a desired final depth into the soil, and, after reaching the final depth, the depth vibrator is drawn out of the ground vibrating at vibration intervals by a defined amount in order to compact the ground stepwise in depth layers, wherein during the vibrating in at the respective compaction depth the method is carried out according to at least one of the abovementioned implementations, wherein the soil stiffness rates determined in the respective compaction depths, at which compaction is terminated, being detected and stored as data pairs comprising a depth value and an associated soil stiffness value.

This mentioned method allows to use the data obtained during the initial compaction detection for other compaction bodies to be created. With this, further compaction bodies of a compaction field to be created can be constructed particularly quickly. By recording and storing the soil stiffness and/or the soil stiffness rate relative to the depth, which are obtained using the above method for online compaction detection, these data can be used for subsequent compaction processes. The mentioned method for online compaction detection therefore only needs to be carried out with a partial number of several compaction columns to be created. Further compaction bodies can be constructed on the basis of soil stiffness and/or soil stiffness rate values previously recorded above depth and used as termination criteria for the respective depth.

In particular, it is provided that at least one second compaction body is created by using the soil stiffness rates determined during the creation of the first compaction body at the respective compaction depths, at which compaction has been completed in each case, for controlling the compaction process for creating the second compaction body. A compaction body can also be referred to as a compaction column or soil column.

The construction of a second soil column is preferably carried out, as is the construction of a first column, by vibrating in the depth vibrator to the desired final depth and subsequent gradual compaction in partial sections until the soil surface is reached. The final depth is the lowest compaction point, which later forms the lower end of the constructed compaction column. For example, the amount by which the depth vibrator is pulled in steps from the final depth to the soil surface for gradual compaction can be between 0.25 and 1.5 m each.

### DESCRIPTION OF THE DRAWINGS

Other objects, features, and advantages of the present invention will become more apparent from the following detailed description of the preferred embodiment and certain modifications thereof, in which:

FIG. 1 shows an exemplary depth vibrator suitable for the implementation of the present method for compaction detection and control as well as for the construction of a compaction column;

FIG. 2 shows schematically a method for manufacturing a compaction column with online compaction detection and control according to the invention;

FIG. 3 shows an arrangement with depth vibrators for compacting soil of a field to be compacted with a schematically drawn model for describing the dynamic conditions during vibratory operation; and

FIG. 4 shows a graphical representation of various characteristic values over time during the implementation of the inventive method in a depth stage.

### DETAILED DESCRIPTION

The present invention is a method for compaction detection and control when compacting a soil by a depth vibrator, and a method for soil improvement using a depth vibrator that is carried out using data from said method for compaction detection and control.

For purposes of description the following reference numerals are assigned to the following features:

### REFERENCES

2 depth vibrator
3 mass bodies
4 vibrator housing
5 rotary drive unit
6 sensor/angle encoder
7 clutch
8 linkage
9 water flushing
10 water flushing
11 pipe
12 sensor/water pressure sensor, water flow meter
13 sensor/acceleration sensor
14 sensor/acceleration sensor
15 tip
16 vibration displacement
17 ground
18 compaction body
19 force sensor
20 compression grid
21 sensor/acceleration sensor
22 spring-damper system with modally resonating ground mass
23 Sensor/displacement transducer
A amplitude
A∞ amplitude at infinitely high excitation frequency
B axis of rotation
C1 compaction, grain rearrangement
C2 grain tension
c damping coefficient
e eccentricity of the imbalance
E plane
k soil stiffness index
k′ soil stiffness increase rate
s1, s2, s3 operating phases
t time
T depth
m Mass of the imbalance
M modal vibrator mass
ΔM modal resonating soil mass
P transition point
V amplification factor
β frequency ratio
φ forward angle
ω exciter frequency

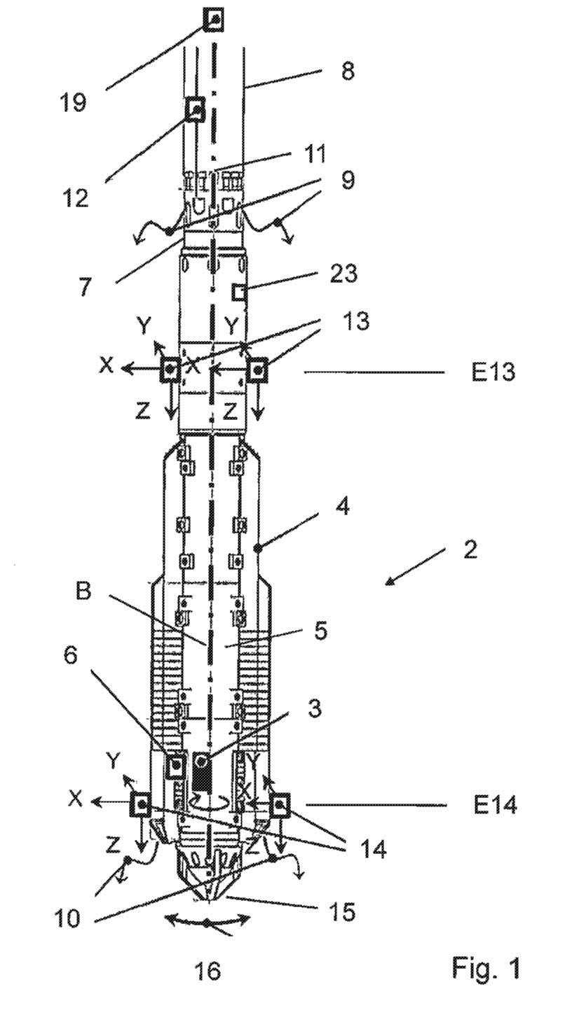

Referring now to FIG. 1, a depth vibrator 2 is used for compacting soil by means of an imbalance. An imbalance is defined as a rotating body 3 which mass is not rotationally symmetrically distributed. For example, the mass inertia axis of the mass body 3 is offset from the rotation axis B, so that the imbalance generates vibrations during rotation, with which the soil and possible feed material are compacted. The pressure vibration compaction process is based on the effect that the friction between the soil grains is temporarily eliminated by the vibration of the depth vibrator 2 and existing pore spaces are closed almost up to the densest packing due to gravity. Depending on the condition of the soil and the amount of compaction required, a reduction in volume may occur.

Accordingly, an exemplary depth vibrator 2 suitable for the implementation of the method for compaction detection and compaction control according to the invention generally comprises the rotationally drivable mass body 3, which is rotationally drivable about the axis of rotation B in a vibrator housing 4. The mass body 3 can be driven by a rotary drive unit 5, for example an electric motor, via a drive shaft (not shown). A position signal representing the position of the imbalance 3 can be detected by a corresponding sensor 6.

The depth vibrator 2 can be suspended from a linkage 8 via a flexible coupling 7. Sinking and/or compaction can optionally be facilitated by one or more water flushes 9, 10 via pipes 11 integrated in the linkage 8. The water flow rate and/or water pressure can be measured using appropriate sensors if necessary and can be controlled accordingly.

First acceleration sensors 13 are provided in a first plane E13 of the depth vibrator 2, particularly above the imbalance 3, and the second acceleration sensors 14 are provided in a second plane E14, particularly below the imbalance 3. The acceleration sensors 13, 14 are used to measure the acceleration of the depth vibrator 2 during the vibration process. Bidirectional acceleration measurements in two planes E13, E14 of vibrator 2 can be used to determine the horizontal motion behaviour at any point of the vibrator, e.g. at the tip 15 or in the position of excitation by imbalance 3. In particular, the vibration displacement 16 at the vibrator tip 15 corresponds to twice the vibration displacement amplitude.

Force sensors 19 may also be provided to detect the suspension force of the vibrator 2 or to determine the tip pressure of the vibrator. In addition, at least one sensor 23 can be provided to measure the penetration depth T of the depth vibrator 2.

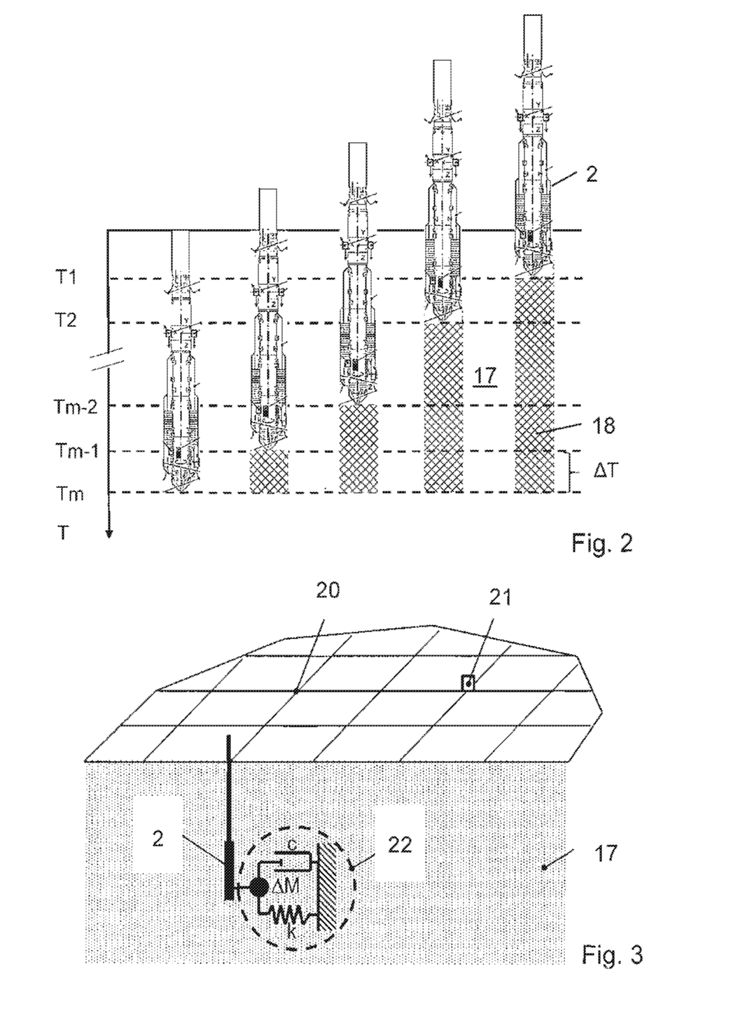

In operation, with reference to FIG. 2, the depth vibrator 2 is first sunk into the ground 17 to the intended improvement depth Tm, if necessary with the addition of water. Subsequently, the depth vibrator 2 is pulled step by step to lesser depths (Tm−1, Tm−2 . . . T2, T1) in order to compact the next soil section above. The depth vibrator 2 can be vibrated into deeper ground areas under tamping motions. In total, a compaction body 18 will be constructed step by step. In particular, for the most accurate compaction detection possible, it is intended that the lifting measure (ΔT) which the depth vibrator 2 is drawn in each case in steps to construct superimposed compaction sections is smaller than or equal to the distance dimension formed between the two planes E13, E14 of the acceleration sensors 13, 14. During step-by-step compaction, a stiffness parameter k representing the soil stiffness is determined over time t for each compaction step. A total stiffness profile k(T) of the soil 17 relative to the depth T can be derived from a large number of individual stiffness characteristic values k determined relative to the depth T of the compaction body 18, in particular from characteristic stiffness characteristic values calculated relative to the depth. The stiffness profile k(T) can, for example, be stored using a suitable data memory and used for the compaction of further columns in a compaction field.

A special feature of the present method for compaction detection or compaction control is that the soil stiffness signals k continuously determined over time can be compared with at least one soil stiffness signal k determined at a previous point in time and from this a soil stiffness increase rate k′ can be derived. This rate of increase k′ indicates by how much, if any, the stiffness of the soil has increased compared with a previously determined value. By comparing the currently determined soil stiffness increase rate k′ with one or more previously determined increase rates k′, conclusions can be drawn about the degree of compaction of the soil section to be compacted. In particular, it can be seen when the increase in the stiffness of the soil increases by leaps. In accordance with the invention, it is provided that the vibration method is terminated at a respective compaction depth (Tm, Tm−1 . . . T1) if the currently determined soil stiffness increase rate k′ exceeds at least one soil stiffness increase rate k′ determined at a previous point in time by a predetermined factor.

The choice of factor can be determined according to the specific technical requirements of the compaction column to be constructed and/or the soil conditions. For example, it may be provided that compaction is interrupted at the respective compaction depth (Tm, Tm−1, . . . , T1) if the determined soil stiffness increase rate k′ is greater than 1.5 times, in particular greater than 2.0 times, optionally greater than 5.0 times, one or more soil stiffness increase rates determined at a previous time. Such a significant increase in soil stiffness of at least 1.5 times suggests that the greatest possible compaction has been achieved.

The soil stiffness parameter k is calculated in particular on the basis of at least the forward angle φ, by which the imbalance mass 3 advances in relation to the direction of motion of the vibrator housing 4 during the vibratory motion, the modal mass M of the depth vibrator 2, a soil mass parameter ΔM representing the soil mass resonating at the depth vibrator 2 and the vibration amplitude A of the depth vibrator 2. The vibrator mass M can be determined by amplitude measurement on the depth vibrator 2 before penetration into soil 17. The determination of the modal total mass from vibrator mass M and modal resonating soil mass ΔM can be determined in the vibrated-in state during operation, in particular in operating phases with a forward angle φ of approximately 180°, which should cover a range of 180°±10°.

Preferably, the soil stiffness signal k representing the soil stiffness of soil 17 is determined taking into account an amplification factor V or a reference amplitude. As a reference amplitude the amplitude of the vibrator 2 at a certain excitation frequency ω while free oscillation can be used. In particular, the measured vibration displacement amplitude A is converted to the amplitude at a theoretically infinitely high excitation frequency A∞ to calculate the amplification factor V:

$$V = \frac{A}{{A}_{∞}} = \frac{A \cdot ( M + Δ M )}{m \cdot e}$$

The dynamic soil stiffness k can then be determined from the measured values and the exciter circular frequency ω in particular with the following formula:

$$k = m \cdot e \cdot {ω}^{2} \cdot ( \frac{1}{{A}_{∞}} - \frac{1}{A} \cdot \frac{sign ( ϕ - 90 ° )}{\sqrt{1 + {tan}^{2} ϕ}} )$$

With the frequency ratio β

$$β = {( 1 - sign ( ϕ - 90 ° ) \cdot ( \frac{{A}_{∞}}{A} ) \cdot {( 1 + {tan}^{2} ϕ )}^{- \frac{1}{2}} )}^{- \frac{1}{2}}$$

the associated Lehr damping factor D can also be determined:

$$D = tan ( ϕ - \frac{sign ( ϕ - 90 ° ) + 1}{2} \cdot 180 ° ) \cdot \frac{1 - {β}^{2}}{2 \cdot β}$$

In this way it is possible to continuously determine the condition-dependent soil stiffness and the damping effect of soil 17 during the penetration of the depth vibrator 2 into the soil or during the compaction process.

It goes without saying that the calculation of a characteristic value representing the soil stiffness is not limited to the described possibility, but that in principle other calculation methods can also be used to determine the soil stiffness characteristic value.

The mechanical model of a harmoniously excited spring/damper system 22, as shown in FIG. 3, can also be used as the basis for the determination of the soil stiffness for the vibration method using a depth vibrator 2. ΔM is the modal resonating soil mass, k is the spring stiffness or soil stiffness and c is a damping coefficient. Further, a compaction grid 20 on the soil surface and a sensor 21 for measuring surface waves are visible. Measurements of surface waves on the soil around a compaction point can provide information about the dynamic vibrator-soil interaction system 22.

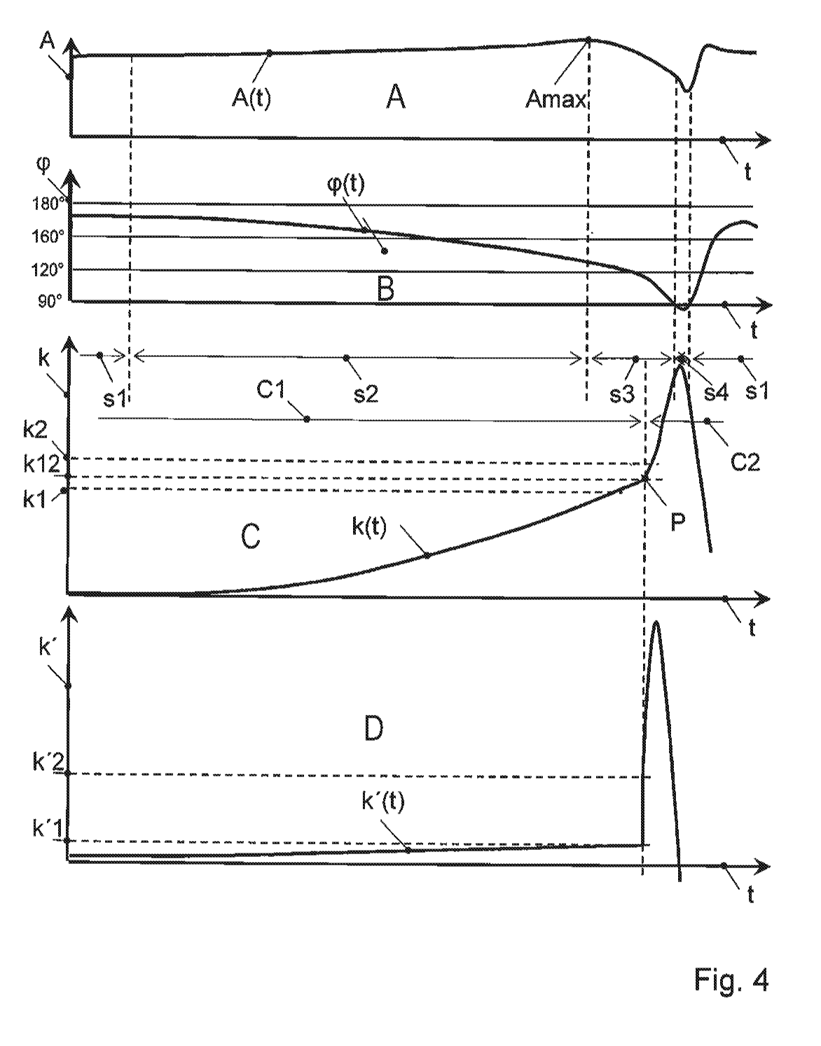

FIG. 4 shows various parameters of compaction by means of a depth vibrator 2 over time for an exemplary compaction process in a compaction section. The upper graph (A) shows the oscillation amplitude A, the second graph (B) the forward angle φ, the third graph (C) the soil stiffness k and the fourth graph (D) the derivation k′ of the soil stiffness, in each case over the time t or in different operating phases s1, s2, s3. Therein, s1 denotes the operating phase of lifting (pulling) the depth vibrator 2 from a deeper depth Tm to the current depth (Tm−1). In the following step, which is marked s2, the vibration in takes place into the soil section to be compacted. The soil 17 is compacted by grain rearrangement, whereby a permanent increase in stiffness is achieved by reducing the pore volume in soil 17. It can be seen that the soil stiffness parameter k rises steadily over time t during vibration and compaction in the operating phase s2. This means that the slope of the soil stiffness parameter k over time is almost constant or increases slightly.

With increasing compression the vibration amplitude A of the depth vibrator 2 increases slightly until a maximum amplitude Amax is reached at resonance. From this point, the operating phase s3 post resonance is available. The soil stiffness k is further increased with an essentially constant gradient as long as the soil is further compacted by grain rearrangement. If maximum compaction is reached, the soil stiffness k(t) increases in a kink, which is marked here as point P. The soil stiffness k(t) increases in a kink. The associated soil stiffness transition value k12 is between a first soil stiffness value k1 with a smaller gradient and a larger soil stiffness value k2 with a larger gradient. According to the kink in point P of curve k(t), the derivation or gradient rate k′ of the soil stiffness rises sharply here. This bend point P marks the transition from compaction C1 by grain rearrangement to grain tension C2, which causes a temporary increase in stiffness due to the currently increased vertical loading of soil 17 by vibrator 2. This temporary increase in the stiffness of soil 17 leads to a significantly higher slope of the calculated soil stiffness parameter k from this transition point P. Subsequently, the soil stiffness parameter k increases further with an almost constant gradient until “overpressing” is achieved in the operating phase s4. In the subsequent step s1, the vibrator 2 is lifted again so that the stiffness parameter k drops again.

It can also be seen that the forward angle φ decreases slowly during compaction C. The forward angle φ decreases slowly during compaction. At the beginning of compaction, the forward angle φ is just below 180° and drops to about 120° until the transition point P is reached. As soon as the transition soil stiffness value k12 is reached, the forward angle φ decreases more steeply, except for about 90°, which are applied in the overpressed state. When s1 is lifted again, the phase angle returns to the initial value.

According to the present method it is intended that the reaching of the transition soil stiffness value k12 or the abrupt increase of the slope of the soil stiffness value k is used as termination criterion for terminating the vibrating process at the respective compaction depth. This results in a particularly efficient compaction process, since on the one hand too little compaction and on the other hand too long a dwell in a compaction stage without additional compaction success is avoided.

It should now be apparent that the foregoing description provides an improved method for compaction detection and control when compacting a soil using a depth vibrator, as well as an improved method for soil improvement using a depth vibrator and the method for compaction detection and control described herein.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept.

We claim:

1. A method for compaction of soil by means of a depth vibrator comprising an imbalance rotationally driveable in a vibrator housing and at least one sensor, the method further comprising the steps of:
inserting the depth vibrator into the soil to a desired final depth (Tm);
compacting the soil by the depth vibrator in a series of compaction steps, wherein during the compaction to a respectively measured depth (T) a forward running angle (φ) of the imbalance and a vibration amplitude (A) of the depth vibrator are determined;
during a compaction step, detecting a soil stiffness profile comprising soil stiffness values (k) determined over time (t) on the basis of said forward running angle (φ) and vibration amplitude (A);
determining a first soil stiffness value (k1) and a second soil stiffness value (k2) from the soil stiffness profile for which a rate of increase (k′2) of the second soil stiffness value (k2) exceeds a rate of increase (k′1) of the first soil stiffness value (k1) by a defined factor;
calculation of a transition soil stiffness value (k12) that is between said first soil stiffness value (k1) and said second soil stiffness value (k2);
storing the transition soil stiffness value (k12) recorded in the respective compaction step to the corresponding depth (T).

2. The method of claim 1 further comprising a step of terminating a compaction step when the rate of increase (k′2) of the second soil stiffness value (k2) is greater than 1.5 times the rate of increase (k′1) of the first soil stiffness value (k1).

3. The method of claim 2, wherein said step of terminating a compaction step comprises terminating said compaction step when the rate of increase (k′2) of the second soil stiffness value (k2) is greater than twice the rate of increase (k′1) of the first soil stiffness value (k1).

4. The method of claim 1, wherein after completion of compaction at a compaction depth (T) the depth vibrator is pulled to the next stepwise depth to be compacted.

5. The method of claim 1, wherein said at least one sensor comprises at least one acceleration sensor configured to measure acceleration of the depth vibrator during compaction.

6. The method of claim 5, wherein said at least one sensor comprises at least one position sensor configured to detect a signal representing the position of the imbalance mounted on the depth vibrator.

7. The method of claim 5, wherein said at least one acceleration sensor comprises a plurality of acceleration sensors mounted in different planes on the depth vibrator.

8. The method of claim 1, wherein said step of determining the first soil stiffness value (k1) and second soil stiffness value (k2) comprises a calculation based on modal resonating soil mass (ΔM).

9. The method of claim 8, wherein said step of determining the first soil stiffness value (k1) and second soil stiffness value (k2) comprises calculating based on the imbalance (m·e), oscillation amplitude (A) and mass (M) of the depth vibrator.

10. The method of claim 9, wherein said calculation comprises a formula:
$$Δ M = \frac{m \cdot e}{A} - M$$

11. The method of claim 1, wherein said step of determining the first soil stiffness value (k1) and second soil stiffness value (k2) comprises a calculation based on measured amplitude (A) and a reference amplitude (A∞) of the vibrator at free oscillation.

12. The method of claim 11, wherein said step of determining the first soil stiffness value (k1) and second soil stiffness value (k2) comprises a calculation according to the formula:
$$V = \frac{A}{{A}_{∞}} = \frac{A \cdot ( M + Δ M )}{m \cdot e}$$
where
V is the amplification factor,
A is the vibration amplitude of the depth vibrator during compaction,
A∞ is the vibration amplitude of the depth vibrator while free oscillation or with an excitation frequency towards infinity,
M is the mass of the depth vibrator,
ΔM is the modal resonating soil mass,
m is the imbalance mass, and
e the eccentricity of the rotating imbalance m relative to the rotary axis.

13. The method of claim 1, wherein a forward angle (φ) of the imbalance relative to the vibrator motion is set to a value greater than 90° and less than 180°.

14. The method of claim 13, wherein the forward angle (φ) of the imbalance relative to the vibrator motion is set to a value greater than 100° and less than 170°.

15. The method of claim 1, wherein said step of determining the first soil stiffness value (k1) and second soil stiffness value (k2) comprises a calculation according to the formula:
$$k = m \cdot e \cdot {ω}^{2} \cdot ( \frac{1}{{A}_{∞}} - \frac{1}{A} \cdot \frac{sign ( ϕ - 90 ° )}{\sqrt{1 + {tan}^{2} ϕ}} )$$
where
A is the vibration amplitude of the depth vibrator during compaction,
A∞ is the vibration amplitude of the depth vibrator while free oscilation or with an excitation frequency towards infinity,
m is the imbalance mass,
e is the eccentricity of the rotating imbalance m relative to the axis of rotation,
ω is the angular frequency of the rotating imbalance, and
φ is the phase advance of the rotating imbalance m relative to motion of the mass M of the depth vibrator.

16. A method for improving the ground by a depth vibrator, comprising the steps of:
establishing a first compaction body by insertion of the depth vibrator into the ground to a desired final depth (Tm);
after reaching the final depth (Tm) with the depth vibrator, stepwise vibration and removal of said vibrator out of the ground in vibration intervals of a defined amount (ΔT), thereby compacting the subsoil step by step in depth sections,
performing the method of claim 1 during vibration of the vibrator at each compaction depth section.

17. The method of claim 16, wherein a soil stiffness profile derived from transition soil stiffness values (k12) detected over the depth (T) is used for controlling construction of at least one compaction body.

18. The method of claim 16, wherein said step of performing the method of claim 1 during vibration of the vibrator at each compaction depth section comprises terminating compaction at said compaction depth section based on a transition soil stiffness value (k12) associated with the respective compaction depth section.

19. The method of claim 17, wherein said soil stiffness profile is used for grid optimization of a grid of a plurality of compaction bodies to be constructed.

20. A method for controlling compaction of soil with an eccentric-weight depth vibrator comprising the steps of:
inserting the depth vibrator into the soil to a desired depth;
compacting the soil at said depth by the depth vibrator;
during said compaction step, detecting a soil stiffness profile comprising a plurality of soil stiffness values measured over time;
determining a soil stiffness value in said profile beyond which further vibration markedly increases subsequent soil stiffness values measured over time, said determined soil stiffness value representing a point at which said soil is as compacted as possible.

21. The method of claim 20, wherein said step of determining a soil stiffness value in said profile beyond further comprises measuring rate of increase of successive soil stiffness values and determining a time at which said rate of increase markedly rises.
